(19) United States
(12) Patent Application Publication
(10) Pub. No.: US 2025/0237582 A1
(43) Pub. Date: Jul. 24, 2025

(54) AIR SAMPLING WITH SYRINGE DEVICE AND METHOD

(71) Applicant: Merit Laboratories, Inc., East Lansing, MI (US)

(72) Inventor: Isaak Murshak, East Lansing, MI (US)

(73) Assignee: Merit Laboratories, Inc., East Lansing, MI (US)

(21) Appl. No.: 19/035,001

(22) Filed: Jan. 23, 2025

Related U.S. Application Data
(60) Provisional application No. 63/624,048, filed on Jan. 23, 2024.

(51) Int. Cl. G01N 1/22; G01N 1/24; G01N 33/00

(52) U.S. Cl. CPC G01N 1/2294; G01N 33/0011; G01N 33/0047; G01N 2001/247

(57) ABSTRACT

A sampling device for assisting with an air sampling process that includes a base forming a channel configured for fitting a syringe. The syringe includes a plunger within a barrel and a hub configured to extend out from the base to access a sorbent tube. A lever is provided that is configured to initiate movement of the plunger. A clamp is connected to the lever and positioned within the channel. The clamp is configured to secure a flange and pull the plunger back when the lever is moved back. A guide bridge extends from a front end of the base and forms an opening for the syringe within the channel and limits undesired movement and/or displacement during use. A restrictor is configured to connect to the sorbent tube and defines an orifice to limit the volume and flow rate of sample being pulled into the sorbent tube.

### CROSS-REFERENCE TO RELATED APPLICATIONS

This application claims priority to U.S. provisional application No. 63/624,048 filed Jan. 23, 2024, and titled “AIR SAMPLING WITH SYRINGE DEVICE AND METHOD”, the subject matter of which is incorporated by reference in its entirety.

### BACKGROUND

For air sampling, thermal desorption tubes or sorbent tubes are often used for capturing contaminants in air. For example, they are used in air sampling for finding longer-chain semi-volatile organic compounds (SVOC) that cannot be recovered from a summa canister. These include acenaphthene, methylnaphthalenes and other Polycyclic Aromatic Hydrocarbons (PAH) with heavy molecular weights.

The sorbent tube is connected to a “sample train” that includes some sort of air sampling pump connected at one end and a hose attachment at the other end to access a target sample area. The pump delivers a pulling force that extracts air and any contaminants that are trapped within the sorbent tube. Forming the necessary pulling force for effective sampling of air can be a challenge. Pumps can be inconvenient and will fail over time. Manual force can be inconsistent and may require exhaustion or fatigue. Moreover, it is difficult to determine the volume obtained. Accordingly, a need exists for a better sampling device and process to ensure safe, effective, and accurate compliance with sampling methods.

### SUMMARY

The present disclosure provides for a sampling device for assisting with an air sampling process. In an example, the device includes (a) a channel formed in a base configured to support a syringe, (b) a lever and a clamp extending from the base configured for actuating the syringe, (c) a lever stop positioned within the channel and protruding outwardly to engage the lever and configured to prevent the lever from moving forward, and (d) a restrictor configured to connect to a sampling component, the restrictor defining an orifice to regulate flow rate and volume of a desired sample entering the sampling component, where the lever is configured to create a pulling force on the sampling component. The syringe may include a barrel and a plunger within the barrel and a hub configured to extend out from the base in a linear direction to access the sampling component. The syringe can be configured to pull 150 ml volume of sample.

The sampling device may also include where the sampling component is a sorbent tube configured to form a seal with the syringe during use. The base can be configured to rest on a flat surface to stabilize the syringe during use. The base and syringe can be configured to engage a sorbent tube for pulling a SVOC or VOC sample from soil. The syringe is configured to fit and slide within the channel.

The sampling device may further include a guide structure extending from the base and defining a stabilization pathway for the syringe to restrict lateral or rotational displacement during operation. The level stop can be configured to engage a locking feature of the lever and is detachable. The channel can be a linear channel and the lever can be configured to cause linear motion of a plunger of the syringe. The clamp can be configured to engage a flange of a plunger of the syringe. The lever can be connected to the clamp by a connector configured to coordinate motion and facilitate uniform actuation of a plunger of the syringe.

The sampling device may also include where the lever stop is positioned to interact with a locking feature on the lever configured for preventing undesired forward movement of the lever and enabling control over the syringe. The sampling device may further include a coupling interface configured to facilitate secure attachment of a hub of the syringe to a sampling component. The pulling force can be a vacuum force. Other technical features may be readily apparent to one skilled in the art from the following figures, descriptions, and claims.

In another example, the present disclosure provides for a method of air sampling using a sampling component, the method includes (a) providing an air sampling device includes: (i) a channel formed in a base configured to support a syringe, (ii) a lever extending from the base, a clamp connecting the lever and the syringe, (iii) a lever stop positioned within the channel and protruding outwardly to engage the lever and prevent the lever from moving forward, (iv) a restrictor defining an orifice to regulate flow rate and volume of a desired sample entering a sampling component, where the lever is configured to create a pulling force on the sampling component, (b) connecting the restrictor to the sampling component forming a sample train, (c) mounting the sampling component to the syringe, (d) mounting the syringe within the channel of the base with the hub extending outwardly opposite the lever allowing the sampling component to access a target sampling area, (e) clamping the syringe with the clamp to secure the syringe within the channel, (f) initiating movement of the lever to actuate the syringe, (g) engaging the lever stop with a locking tab of the lever to prevent the lever from moving forward once pulled back forming a pulling force at an opening of the hub and the sampling device, (h) allowing the device to pull sample into the sampling component for a predetermined period of time sufficient to obtain a desired volume of sample. The method may also include where the base further includes a guide structure extending from the base and defining a stabilization pathway for the syringe to restrict lateral or rotational displacement during operation, and further includes the steps of removing the sampling device from the sample area and disconnecting the syringe from the base. Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a sampling device of the present disclosure with a syringe and a lever in a closed position (i.e., a section position).

FIG. 2 illustrates the sampling device of FIG. 1 with the syringe connected to a sorbent tube and the level in an open position.

FIG. 3 illustrates a magnified view of the open position of the lever from FIG. 2.

FIG. 4 illustrates an opposite side view of the open position of the lever from FIG. 3.

FIG. 5 illustrates a magnified top side view of the open position of the lever from FIG. 4.

FIG. 6 illustrates a top side front view of the open position of the lever from FIG. 5.

FIG. 7 illustrates a top side view of a closed position of the lever of sampling device of the present disclosure.

FIG. 8 illustrates a photograph of a sorbent tube and a sample train according to the present disclosure.

FIG. 9 illustrates a method 900 in accordance with a sampling method of the present disclosure.

### DETAILED DESCRIPTION

In an example sampling method, a syringe is used (i.e., as the method pump) to draw soil vapor or air through a sampling component like a thermal desorption tube or a sorbent tube. Soil vapor is an understood term in the environmental field that generally means vapor coming from the soil and is not collected in a closed system. Sorbent tubes are widely used collection media for sampling hazardous gases and vapors. They can be made of metal or glass and contain various types of solid adsorbent material (sorbents). Commonly used sorbents include activated charcoal, silica gel, and organic porous polymers such as Tenax and Amberlite XAD resins. Solid sorbents are selected for sampling specific compounds in air because they trap and retain the compound(s) of interest even in the presence of other compounds. They generally do not alter the compound(s) of interest and allow collected compounds to be easily desorbed or extracted for analysis. Sorbent tubes are attached to air sampling pumps for sample collection. A pump with a calibrated flow rate in ml/min draws a known volume of air through the sorbent tube. Sometimes, pumps and sorbent tubes are placed in areas for fixed-point sampling. Chemicals are trapped onto the sorbent material throughout the sampling period.

Occasionally, when desorbing the air sample from the sorbent tube, a large portion of the analyte will fail to go into the solution or other desorbent fluid. In these cases, the sorbent tubes will have to be adjusted for desorption efficiency (DE). Suitable sorbent tubes may be prepacked and may include a multi-layered sorbent packed tube such as a mix of Carbograph 2 60/80; Carbograph 1 40/60; Carbosive SIII 60/80.) A sample train is formed that includes the sorbent tube connected to a syringe for forming a pulling force to extract air from a sampling target area, along with a union or fitting to connect the restrictor and the sorbent tube. A continuous fluid (or gas) communication pathway is formed from the external (sampling) environment, the restrictor, the sorbent tube, and the syringe, for example with or without any intervening fluid flow components (e.g., tubing or fitting) between any of the foregoing sampling train components.

In an example sampling method, a maximum flow rate of 200 ml/minute is recommended in order to minimize the risk of leaks around the probe annulus as well as minimize the vacuum imposed on the soil and stripping of SVOCs from the soil or free product. This restriction is achieved by placing a suitable restrictor inside the sample train that will only allow for a maximum sampling rate of about 188 ml/min. The restrictor defines a smaller orifice that forms a maximum flow rate limited by sonic velocity or critical velocity (e.g., 188 ml/min; maximum velocity obtainable by a compressible fluid going through a 0.006-in orifice from 1 atm to vacuum) resulting from the pressure differential and the orifice size. Critical velocity is the speed and direction at which the fluid can flow through a conduit without becoming turbulent. The orifice limits air to pass from the sample area and through the sorbent tube. The restrictor can be a fitting that fits within the sorbent tube and thus restricts the velocity of air that passes through the sorbent tube. This velocity or sampling rate is within sampling standards according to EPA Method TO17, Section 6.3.1, which states: “sampling tubes capable of independent control of sampling rate at a settable value in the range 10 to 200 mL/min).” The syringe is a type of manual pump to create the pressure differential to pull the required sample volume. In an example, this pressure differential is the pulling force and it can also create a vacuum. The maximum velocity or sampling rate is thus reached due to the presence of the restrictor (e.g. 188 ml/min; maximum velocity obtainable by a compressible fluid going through a 0.006-in orifice from 1 atm to vacuum).

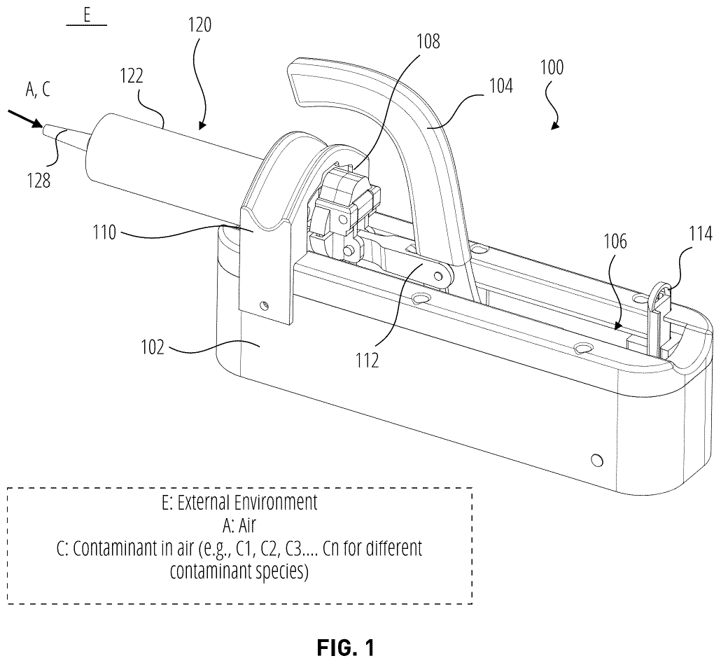

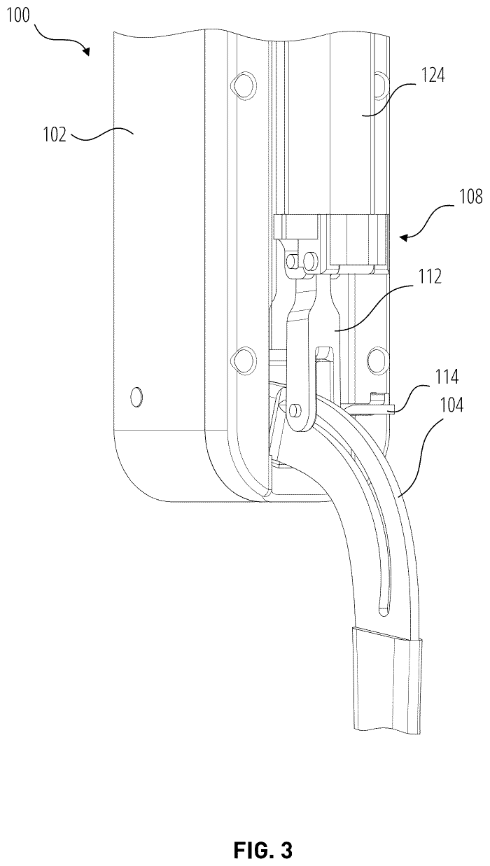

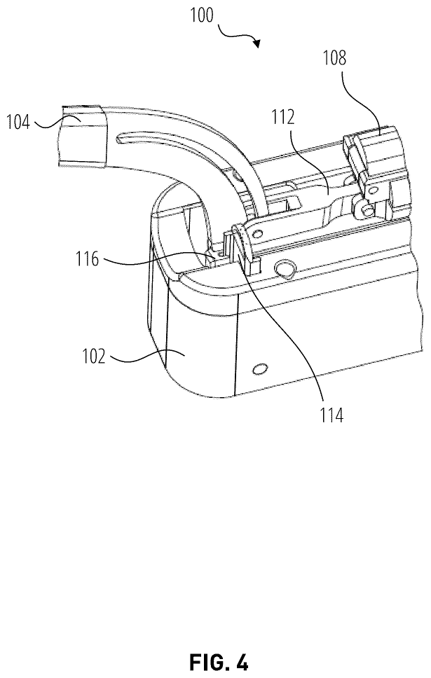

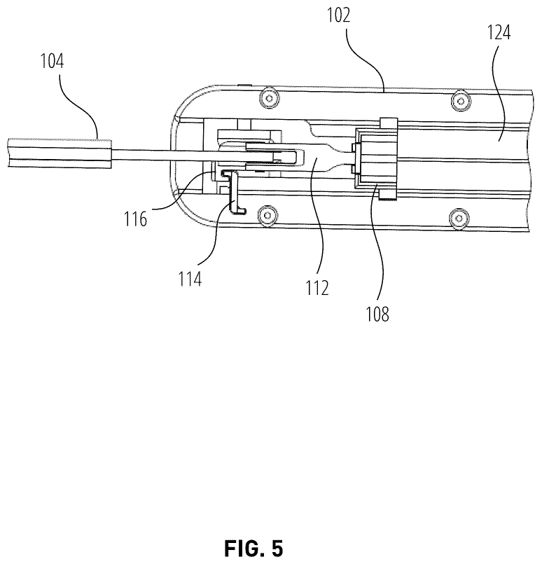

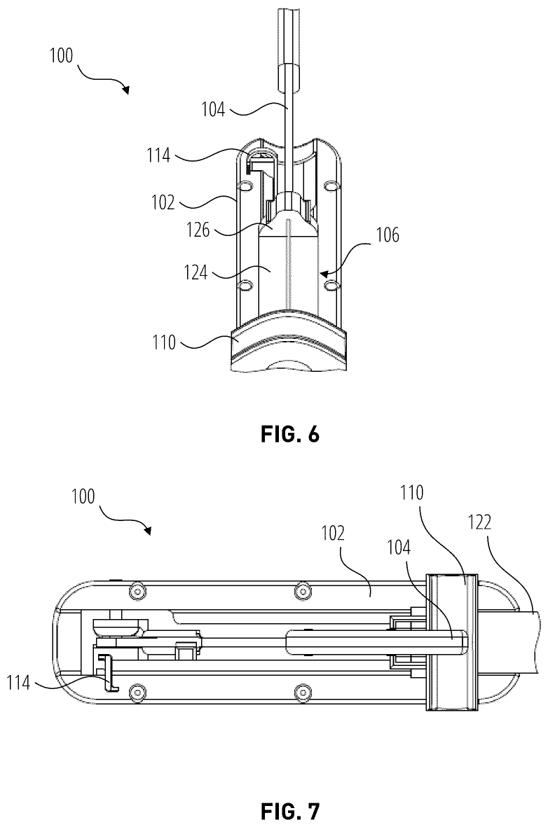

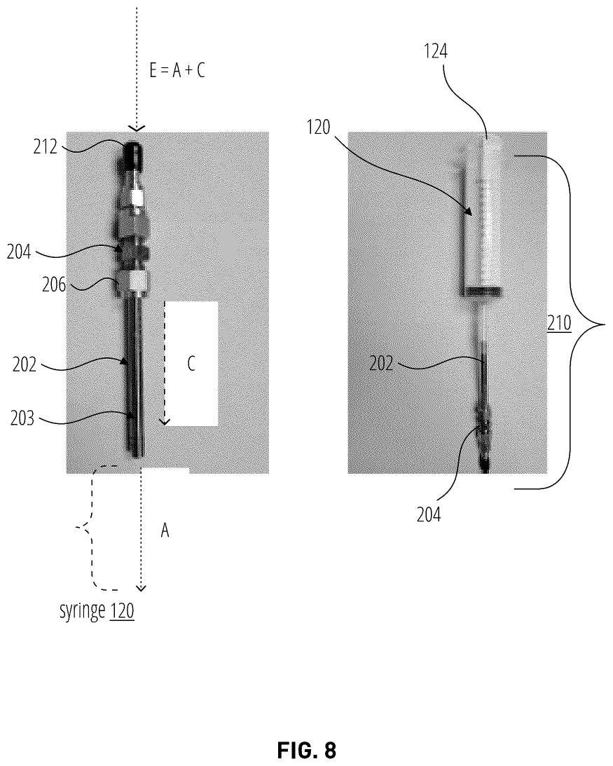

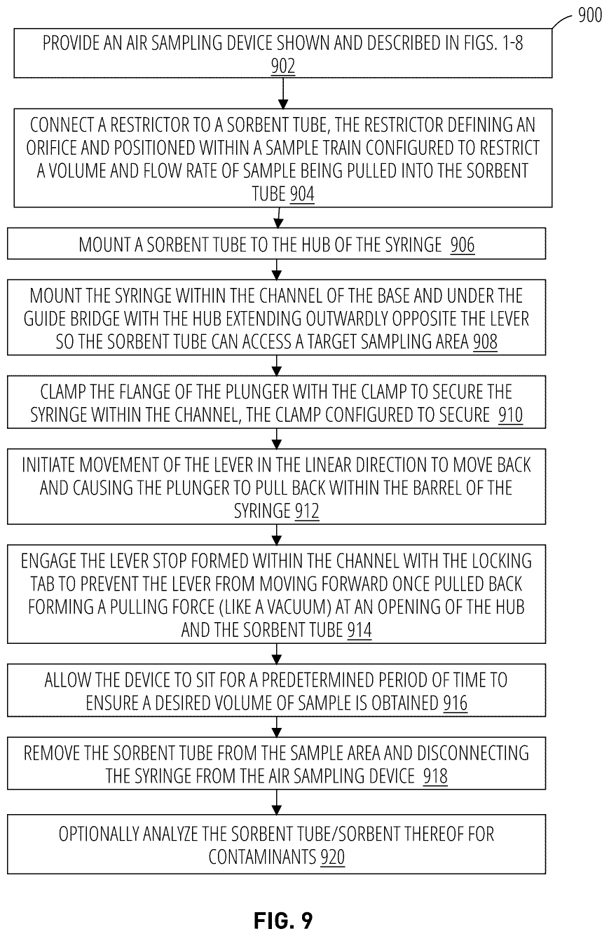

Referring to FIGS. 1-9, the present disclosure provides for a sampling device 100 configured for mounting a syringe 120 and pulling a plunger 124 to form a vacuum for capturing a gas sample within a sorbent tube 202. Syringe 120 includes a barrel 122, plunger 124, and a hub 128. Barrel 122 is generally hollow to allow for the plunger 124 to travel forward and back within barrel 122. Hub 128 can be referred to as a flow-path connector since it connects the flow from the sorbent tube 202 to the barrel 122. Hub 128 defines an opening and allows for air to be pulled into the barrel 122 or pushed out of barrel 122. A plunger 124 is configured to pass through barrel 122 and includes a stopper (not shown) that forms a seal against interior walls of barrel 122. The stopper is typically rubber or silicon, to allow it to move forward and back within barrel 122 while maintaining a seal. Plunger 124 further includes a flange 126 positioned opposite the stopper to allow for pulling the plunger 124 back to initiate a pulling force through the hub 128.

Sampling device 100 includes a base 102 and defines an interior elongated channel 106. A lever 104 is positioned on an opposite end from a guide bridge 110. A clamp 108 is configured to hold the flange 126 of syringe 120 securely. Lever 104 is configured to move forward and back to cause the plunger 124 to move forward and back within the barrel 122. In this example, clamp 108 is connected to lever 104 by a connector 112.

The movement, or rotation, of lever 104 allows the plunger 124 to move linearly within barrel 122. If the hub 128 is connected to another object and forms a seal, the pulling of plunger 124 will form a pulling force within the barrel 122. If the the connection forms an air-tight seal, the pulling force will form a vacuum. The pulling force can cause air or other material to be pulled into the connected sorbent tube 202.

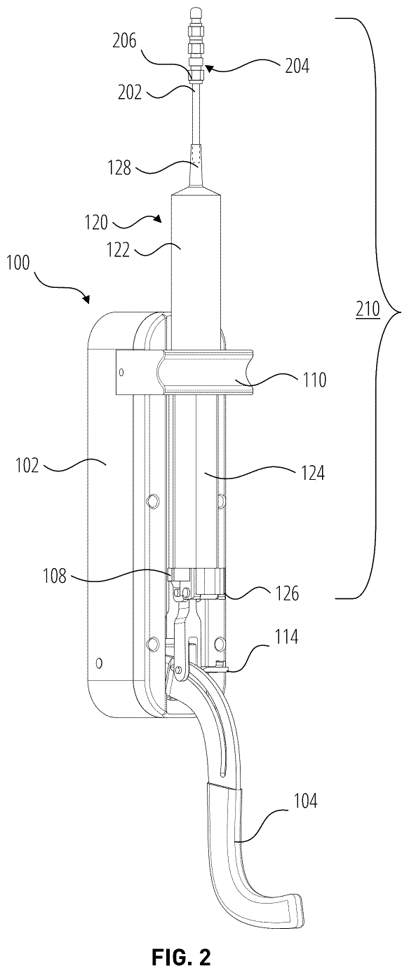

FIG. 2 illustrates a sorbent tube 202 connected to the hub 128 of syringe 120 forming a sample train 210. Sorbent tube 202 is configured to absorb an air sample consistent with EPA sampling requirements. A packed sorbent tube 202 is suitable to trap any contaminant gas species C while preventing other contaminants or solid material from entering the tube 202. When mounted to the hub 128, with the lever 104 pushed forward (i.e., towards the sorbent tube 202), the plunger 124 is fully forward within barrel 122. As lever 104 is pulled or rotated back, a vacuum forms causing air A containing contaminants C therein, if any exist, to be pulled into the sorbent tube 202 from the external environment E. Contaminants C can include different contaminants (e.g., C₁, C₂, C₃ . . . Cₙ) that will be identified during the analysis of the sample. The environment E includes air A plus contaminant(s) C (i.e., E=A+C). As an air sample (A+C) is pulled through the sorbent tube 202, contaminant C is absorbed by the packed sorbent 203 and air A passes through and into the barrel122 of syringe 120.

The sample train 210 further includes a restrictor 204. The restrictor 204 defines a relatively smaller orifice to restrict air flow within the sample train 210. The restrictor 204 is provided and inserted within the sample train 210 to limit the volume and/or velocity of air A that can pass into the sorbent tube 202 and thus, restricts flow rate. In another example, restrictor 204 is connected to sorbent tube 202 by a union fitting 206.

Use of sampling device 100 allows for holding the syringe 120 in place with the plunger 124 pulled back forming the vacuum. The restrictor 204 ensures that a suitable flow rate is achieved for a qualifying sampling process. Forming of a vacuum occurs when the plunger 124 is pulled away from the hub 128 and the sorbent tube 202 is in place forming a seal. However, this pulling motion, when done manually can be a strain on a user as the force is significant while the sorbent tube 202 is filling with the desired sample. Significant strength and endurance may be required, particularly of a user's forearm. Sampling device 100 allows for suitable sampling of a target area without manual/user involvement beyond initial activation and locking of the lever 104.

Base 102 further includes a lever stop 114 that engages a corresponding locking tab 116 extending out from the lever 104. Locking tab 116 protrudes to the side of the linear direction of the lever 104 and channel 106 is configured to allow locking tab 116 to pass through during the forward and back motion of lever 104. Once lever 104 is pulled back to form the vacuum force on sorbent tube 202, the locking tab 116 can be engage with and abut the lever stop 114 to prevent forward movement of plunger 124. This imposes or forces a continuous vacuum force on sorbent tube 202. The vacuum force on sorbent tube 202 allows for the pulling of an air sample at a maximum velocity that is still within guidelines and requirements of the EPA qualifying sampling method.

In an example, lever stop 114 is removable to disengage from a slot formed within base 102 by sliding it out. When lever stop 114 is disengaged, the plunger 124 is allowed to naturally move forward if a vacuum force exists indicating that the sampling event is not yet complete or that the maximum volume has not yet been reached. This can be done at the conclusion of any sampling event. However, if the desired volume is reached and/or maxed out based on the overall syringe volume, meaning it is filled with gas, then the removal of the lever stop 114, by sliding it up and out, will show that the plunger remains in place without being drawn back into the barrel. Accordingly, the lever stop 114 should slide up and out with little force applied since the lever will not be pressing against it. This is an indication that the sampling event has concluded.

Guide bridge 110 prevents syringe 120 from lifting out from sampling device 100 and also may help ensure that it stays in linear position during use. Guide bridge 110 forms an opening over channel 106 to allow the syringe 120 to rest within sampling device 100. Once syringe 120 is placed within the guide bridge 110 and channel 106, a clamp 108 is placed over the flange 126 of plunger 124. In this example, a clamp 108 is shown, however, any connection means for holding the flange 126 of plunger 124 is within the scope of this disclosure.

Syringe 120 can be in a closed position with plunger 124 fully forward towards the hub 128. The sorbent tube 202 is securely positioned within the opening of hub 128. In this closed position, as shown in FIG. 1, there should be no pulling force initiated. As lever 104 is pulled back, as shown in FIG. 2, plunger 124 moves back forming the vacuum force. Once the lever 104 is pulled back, the lever stop 114 can engage the locking tab 116 to prevent linear forward movement. This vacuum force allows sorbent tube 202 to pull a gas sample without manual strain or user involvement to hold sample train 210 in place. If a sampling process requires a specific volume, using a proper syringe volume will allow for that to occur.

The present disclosure provides for a sampling method using sampling device 100 of the present disclosure. A sorbent tube 202 is connected to a syringe 120. A leak-test is performed prior to use to ensure the volume taken is accurate. First, a syringe 120 is selected and an attempt to pull the plunger 124 back slowly is done. The plunger 124 will show complete resistance to movement if the seal is leak-tight. Any backwards motion of the plunger will indicate a leak. This is consistent with EPA Method TO17 Section 8.2.1.2, which states: “The sample integrity protection measures and pre-analysis checks required include: Tube leak testing. This activity must not jeopardize sample integrity and Leak testing of the sample flow path. This activity must not jeopardize sample integrity.”

Once the leak check is complete, a rubber cap 212 of the sorbent tube 202 is removed. The sorbent tube 202, connected to the hub 128 of syringe 120 and a hose or line, forms a soil-gas line once inserted into a target soil area. This can also be used with ambient air or sub-sampling in building to collect the air contained in soil or fill directly under a building's lowest floor, regardless of whether the building has slab-on-grade or basement construction by placing a pin or flow path to that soil. EPA Method TO17 Section 6.5.2 states: “Using clean gloves, remove the sample tubes from the container, take off their caps and attach them to the sampling lines with non-outgassing flexible tubing. Uncap and immediately reseal the required number of field blank tubes.”

In an example, the plunger 124 is then pulled back until a total volume of a desired volume is reached by pulling lever 104. If the method requires to not exceed 150 ml total volume, then the barrel volume (i.e., internal volume defined by barrel 122 with a retracted plunger 124) should include the same volume restriction. The connecting of the sorbent tube 202 to the syringe 120 forms a sample train. The sampling process then includes a step of letting the sample train sit for a period of time, (e.g., about 1 minute) to ensure that enough time has passed to collect the 150 ml sample volume. EPA Method TO17 Section 6.4.1 states: “Select sampling rates compatible with the collection of 1- and 4-liter total sample volume (or of proportionally lower/higher sampling volumes).” Collecting 150 ml sample volume is allowable by the method (See EPA Method TO17, section 6.4.1). In this example, it is important that no more than 150 ml of total sample volume is collected because of the sensitivity of a corresponding analytical machine like a GC/MS used to run TO17 and for possible carryover or (inter-sample contamination) to the next sample. The 150 ml sample volume is large enough to reach the required reporting limits and levels of detection for many environmental agencies and guidance for SVOC analytes in air. This sample volume also allows the option to re-analyze the sorbent tube 202 one or more times, giving plenty of sample volume for a duplicate run. This is an advantage over published methods, since existing methods can only analyze the sample once.

Once the sampling process is complete, the soil-gas line is disconnected and the black rubber cap is placed back onto the sorbent tube 202. EPA Method TO17 Section 6.8 states, “Immediately remove the sampling tubes with clean gloves, recap the tubes with Swagelok® fittings using PTFE ferrules.” The sorbent tube 202 includes a silicone tube that should be disconnected from the sorbent tube 202 and a brass cap should be placed back onto the sorbent tube 202. EPA Method TO17 Section 6.8.1 states, “Immediately remove the sampling tubes with clean gloves, recap the tubes with compression tube fittings using PTFE ferrules.”

Referring to FIG. 9, in block 902, method 900 provides an air sampling device as described with respect to FIGS. 1-8. In block 904, method 900 connects a restrictor to a sorbent tube, the restrictor defining an orifice and positioned within a sample train configured to restrict a volume and flow rate of sample being pulled into the sorbent tube. In block 906, method 900 mounts a sorbent tube to the hub of the syringe. In block 908, method 900 mounts the syringe within the channel of the base and under the guide bridge with the hub extending outwardly opposite the lever so the sorbent tube can access a target sampling area. In block 910, method 900 clamps the flange of the plunger with the clamp to secure the syringe within the channel, the clamp configured to secure. In block 912, method 900 initiates movement of the lever in the linear direction to move back and causing the plunger to pull back within the barrel of the syringe. In block 914, method 900 engages the lever stop formed within the channel with the locking tab to prevent the lever from moving forward once pulled back forming a pulling force (like a vacuum) at an opening of the hub and the sorbent tube. In block 916, method 900 allows the device to sit for a predetermined period of time to ensure a desired volume of sample is obtained. In block 918, method 900 removes the sorbent tube from the sample area and disconnecting the syringe from the air sampling device. In block 920, the captured sample within the sorbent tube is then analyzed for contaminants (e.g., sample analysis).

Referring to Table 1 and Table 2 below, the process and device of the present disclosure is sufficient for testing of list of analytes set forth along with the corresponding CAS numbers. In Table 1, the analytes of interest can be captured using a canister or a tube. In Table 2, the analytes of interest can by captured by tube only because the analytes are either larger, heavier, or both.

TABLE 1

| Analyte | Cas # |
|---|---|
| Acetone | 67-64-1 |
| 1,3-Butadiene | 106-99-0 |
| Benzene | 71-43-2 |
| Bromodichloromethane | 75-27-4 |
| Bromoform | 75-25-2 |
| Bromomethane | 74-83-9 |
| Vinyl bromide | 593-60-2 |
| Benzyl chloride | 100-44-7 |
| Carbon disulfide | 75-15-0 |
| Chlorobenzene | 108-90-7 |
| Chloroethane | 75-00-3 |
| Chloroform | 67-66-3 |
| Chloromethane | 74-87-3 |
| 3-Chloropropene | 107-05-1 |
| 2-Chlorotoluene | 95-49-8 |
| Carbon tetrachloride | 56-23-5 |
| Cyclohexane | 110-82-7 |
| 1,1-Dichloroethane | 75-34-3 |
| 1,1-Dichloroethene | 75-35-4 |
| 1,2-Dibromoethane | 106-93-4 |
| 1,2-Dichloroethane | 107-06-2 |
| 1,2-Dichloropropane | 78-87-5 |
| 1,4-Dioxane | 123-91-1 |
| Dichlorodifluoromethane | 75-71-8 |
| Dibromochloromethane | 124-48-1 |
| trans-1,2-Dichloroethene | 156-60-5 |
| cis-1,2-Dichloroethene | 156-59-2 |
| cis-1,3-Dichloropropene | 10061-01-5 |
| 1,3-Dichlorobenzene | 541-73-1 |
| 1,2-Dichlorobenzene | 95-50-1 |
| 1,4-Dichlorobenzene | 106-46-7 |
| trans-1,3-Dichloropropene | 10061-02-6 |
| Ethanol | 64-17-5 |
| Ethylbenzene | 100-41-4 |
| Ethyl Acetate | 141-78-6 |
| 4-Ethyltoluene | 622-96-8 |
| Freon 113 | 76-13-1 |
| Freon 114 | 76-14-2 |
| Heptane | 142-82-5 |
| Hexachlorobutadiene | 87-68-3 |
| Hexane | 110-54-3 |
| 2-Hexanone | 591-78-6 |
| Isopropyl Alcohol | 67-63-0 |
| Methylene chloride | 75-09-2 |
| 2-Butanone (MEK) | 78-93-3 |
| 4-Methyl-2-pentanone (MIBK) | 108-10-1 |
| tert-Methyl butyl ether (MTBE) | 1634-04-4 |
| Methyl methacrylate | 80-62-6 |
| Naphthalene | 91-20-3 |
| Propylene | 115-07-1 |
| Styrene | 100-42-5 |
| 1,1,1-Trichloroethane | 71-55-6 |
| 1,1,2,2-Tetrachloroethane | 79-34-5 |
| 1,1,2-Trichloroethane | 79-00-5 |
| 1,2,4-Trichlorobenzene | 120-82-1 |
| 1,2,4-Trimethylbenzene | 95-63-6 |
| 1,3,5-Trimethylbenzene | 108-67-8 |
| 2,2,4-Trimethylpentane | 540-84-1 |
| Tert-butyl Alcohol | 75-65-0 |
| Tetrachloroethene | 127-18-4 |
| Tetrahydrofuran | 109-99-9 |
| Toluene | 108-88-3 |
| Trichloroethene | 79-01-6 |
| Trichlorofluoromethane | 75-69-4 |
| Vinyl chloride | 75-01-4 |
| Vinyl acetate | 108-05-4 |
| p,m-Xylene | M/P-XYLENE |
| o-Xylene | 95-47-6 |
| Total Xylenes | 1330-20-7 |

TABLE 2

| Analyte | Cas # |
|---|---|
| Naphthalene | 91-20-3 |
| 2-Methylnaphthalene | 91-57-6 |
| 1-Methylnaphthalene | 90-12-0 |
| Acenaphthene | 83-32-9 |
| Acenaphthylene | 208-96-8 |
| Fluorene | 86-73-7 |
| Phenanthrene | 85-01-8 |
| Anthracene | 120-12-7 |
| Pyrene | 129-00-0 |
| Benzo(a)anthracene | 56-55-3 |

In embodiments, the process and device of the present disclosure can be used to sample, capture, and/or detect polyfluoroalkyl substances (“PFAS”), for example anionic PFAS, cationic PFAS, or both, which can be an SVOC or a VOC. Examples of anionic PFAS include Perfluorobutanoic Acid (PFBA), Perfluoropentanoic Acid (PFPeA), 4:2 Fluorotelomer Sulfonic Acid (4:2 FTSA), Perfluorohexanoic Acid (PFHxA), Perfluorobutane Sulfonic Acid (PFBS), Perfluoroheptanoic Acid (PFHpA), Perfluoropentane Sulfonic Acid (PFPeS), 6:2 Fluorotelomer Sulfonic Acid (6:2 FTSA), Perfluorooctanoic Acid (PFOA), Perfluorohexane Sulfonic Acid (PFHxS), Perfluorohexane Sulfonic Acid-Linear (PFHxS-LN), Perfluorohexane Sulfonic Acid Branched (PFHxS-BR), Perfluorononanoic Acid (PFNA), 8:2 Fluorotelomer Sulfonic Acid (8:2 FTSA), Perfluoroheptane Sulfonic Acid (PFHpS), Perfluorodecanoic Acid (PFDA), N-Methyl Perfluorooctane Sulfonamidoacetic Acid (N-MeFOSAA), N-Ethyl Perfluorooctane Sulfonamidoacetic Acid (EtFOSAA), Perfluorooctane Sulfonic Acid (PFOS), Perfluorooctane Sulfonic Acid-Linear (PFOS-LN), Perfluorooctane Sulfonic Acid-Branched (PFOS-BR), Perfluoroundecanoic Acid (PFUnDA), Perfluorononane Sulfonic Acid (PFNS), Perfluorododecanoic Acid (PFDoDA), Perfluorodecane Sulfonic Acid (PFDS), Perfluorotridecanoic Acid (PFTrDA), Perfluorooctane Sulfonamide (FOSA), Perfluorotetradecanoic Acid (PFTeDA), Undecafluoro-2-methyl-3-oxahexanoic acid (GenX), 10:2-fluorotelomersulfonic acid (10:2 FTS), Perfluorododecanesulfonic acid (PFDoS), Perfluorododecanoic acid (PFDoA), Pedluorohexadecanoic acid (PFHxDA), Pedluorooctadecanoic acid (PFODA), Pedluorotetradecanoic acid (PFTeDA), Pedluorotridecanoic acid (PFTrDA), Pedluoroundecanoic acid (PFUDA), 4,8-dioxa-3H-perfluorononanoic acid (DONA), 9-chlorohexadecafluoro-3-oxanonane-1-sulfonic acid (9Cl-PF3ONS), 11-chloroeicosafluoro-3-oxaundecane-1-sulfonic acid (11Cl-PF3OUdS), Perfluoro (3,4,5,9-tetraoxadecanoic) acid, Perfluoro (3,5,7-trioxaoctanoic) acid, Perluoro (3,5-dioxahexanoic) acid, Perfluoro-2-methoxyacetic acid, Pertluoro-2-methoxyethoxyacetic acid, Perfluoro-3-methoxypropanoic acid, Perfluoro-4-isopropoxybutanoic acid, Perfluoro-4-methoxybutanoic acid, and their corresponding salts. Examples of cationic PFAS include perfluorooctaneamido quaternary ammonium salt (PFOAAmS) and 6:2 fluorotelomer sulfonamido amine (FtSaAm).

It should be noted that the steps described in the method of use can be carried out in many different orders according to user preference. The use of “step of” should not be interpreted as “step for”, in the claims herein and is not intended to invoke the provisions of 35 U.S.C. § 112 (f). Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination, or addition of certain steps, including or excluding certain maintenance steps, etc., may be sufficient.

What is claimed is:

1. A sampling device for assisting with an air sampling process, the device comprises:
(a) a channel formed in a base configured to support a syringe;
(b) a lever and a clamp extending from the base configured for actuating the syringe;
(c) a lever stop positioned within the channel and protruding outwardly to engage the lever and configured to prevent the lever from moving forward; and
(d) a restrictor configured to connect to a sampling component, the restrictor defining an orifice to regulate flow rate and volume of a desired sample entering the sampling component;
wherein the lever is configured to create a pulling force on the sampling component.

2. The sampling device of claim 1, wherein the syringe includes a barrel and a plunger within the barrel and a hub configured to extend out from the base in a linear direction to access the sampling component.

3. The sampling device of claim 2, wherein the lever is configured to initiate movement of the plunger in a linear direction.

4. The sampling device of claim 2, wherein the plunger includes a flange accessible by the clamp to initiate movement of the plunger in a linear direction.

5. The sampling device of claim 1, wherein the syringe is configured to pull 150 ml volume of sample.

6. The sampling device of claim 1, wherein the sampling component is a sorbent tube configured to form a seal with the syringe during use.

7. The sampling device of claim 1, wherein the base is configured to rest on a flat surface to stabilize the syringe during use.

8. The sampling device of claim 1, wherein the base and syringe are configured to engage a sorbent tube for pulling a SVOC or VOC sample from soil.

9. The sampling device of claim 1, wherein the syringe is configured to fit and slide within the channel.

10. The sampling device of claim 1, further comprising a guide structure extending from the base and defining a stabilization pathway for the syringe to restrict lateral or rotational displacement during operation.

11. The sampling device of claim 10, wherein the guide structure is a guide bridge.

12. The sampling device of claim 1, wherein the level stop is configured to engage a locking feature of the lever and the level stop is detachable.

13. The sampling device of claim 1, wherein the channel is a linear channel and the lever is configured to cause linear motion of a plunger of the syringe.

14. The sampling device of claim 1, wherein the clamp is configured to engage a flange of a plunger of the syringe.

15. The sampling device of claim 1, wherein the lever is connected to the clamp by a connector configured to coordinate motion and facilitate uniform actuation of a plunger of the syringe.

16. The sampling device of claim 1, wherein the lever stop is positioned to interact with a locking feature on the lever configured for preventing undesired forward movement of the lever and enabling control over the syringe.

17. The sampling device of claim 1, further comprising a coupling interface configured to facilitate secure attachment of a hub of the syringe to a sampling component.

18. The sampling device of claim 1, wherein the pulling force is a vacuum force.

19. A method of air sampling using a sampling component, the method comprising:
(a) providing an air sampling device comprising:
(i) a channel formed in a base configured to support a syringe,
(ii) a lever extending from the base, a clamp connecting the lever and the syringe,
(iii) a lever stop positioned within the channel and protruding outwardly to engage the lever and prevent the lever from moving forward,
(iv) a restrictor defining an orifice to regulate flow rate and volume of a desired sample entering a sampling component,
wherein the lever is configured to create a pulling force on the sampling component;
(b) connecting the restrictor to the sampling component forming a sample train;
(c) mounting the sampling component to the syringe;
(d) mounting the syringe within the channel of the base with the hub extending outwardly opposite the lever allowing the sampling component to access a target sampling area;
(e) clamping the syringe with the clamp to secure the syringe within the channel;
(f) initiating movement of the lever to actuate the syringe;
(g) engaging the lever stop with a locking tab of the lever to prevent the lever from moving forward once pulled back forming a pulling force at an opening of the hub and the sampling device; and
(h) allowing the device to pull sample into the sampling component for a predetermined period of time sufficient to obtain a desired volume of sample.

20. The method of claim 19, wherein the base further comprises a guide structure extending from the base and defining a stabilization pathway for the syringe to restrict lateral or rotational displacement during operation, and further comprising the steps of removing the sampling device from the sample area and disconnecting the syringe from the base.
